(19) United States
(12) Patent Application Publication
(10) Pub. No.: US 2006/0241501 A1
(43) Pub. Date: Oct. 26, 2006

(54) Method and apparatus for detecting abnormal epithelial tissue

(75) Inventor: Mark Bride, Peoria, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: Zila Pharmaceuticals, Inc.

(21) Appl. No.: 11/400,110

(22) Filed: Apr. 7, 2006

Related U.S. Application Data
(63) Continuation-in-part of application No. 10/564,800, filed on Jan. 12, 2006.

(51) Int. Cl. A61B 6/00

(52) U.S. Cl. 600/476; 600/478

(57) ABSTRACT

A method of detecting abnormal epithelial tissue. The method includes the steps of providing a light source, providing a housing having an opening defined therein, inserting the light source into the housing, illuminating an area of epithelial tissue with incident light emitted from the light source that is directed through the opening in the housing, wherein at least a portion of the incident light is reflected from the area, thereby creating reflected light having has at least one wavelength, and viewing the reflected light. The housing is preferably opaque.

### RELATED APPLICATIONS

This application is a continuation-in-part of application Ser. No. 10/564,800, filed Sep. 28, 2004, the entirety of which is incorporated by reference herein.

### FIELD OF THE INVENTION

This invention relates to methods for detecting abnormal epithelial tissue, which may harbor tumor phenotypes.

In another respect the invention pertains to improved methods for conducting real time in vivo examinations of epithelial tissue to detect abnormalities which may be cancerous or which may eventually develop invasive cancer.

### BACKGROUND OF THE INVENTION

Patients who delay in obtaining a cancer consultation for at least two months have significantly higher relative hazards of death than do patients with a shorter delay. Thus, if patients are more regularly subjected to effective cancer screening, the mortality risks of cancer could be reduced. Thus, there was a need for a simple, rapid screening test for detecting abnormal mucosal tissue which may harbor tumor phenotypes, which may indicate the presence of or the eventual development of invasive cancer.

Abnormal epithelial tissue can be visually identified and located in real time in vivo using selective light examinations, which are admirably suited for rapid and inexpensive screening carried out as routine medical and dental examinations. Illustratively, U.S. Pat. Nos. 5,179,938 and 5,329,938 to Lonky, incorporated herein by reference, describe instruments equipped with a chemiluminescent light source which radiates in the visible green, blue and, optionally, red spectrums, with spectral peaks at 430, 550 and 580 nm (referred to herein as “incident light”). Under such illumination, with normal ambient light suppressed, abnormal mucosal tissue appears white (referee to herein as “reflected light”). Illustratively, such selective light devices for practicing such in vivo examinations are commercially available under the registered trademark VIZILITE® from Zila Pharmaceuticals, Inc., Phoenix, Ariz., USA.

The selective visualization of abnormal mucosal tissue using such light sources is hindered by normal ambient light (daylight or normal artificial light) falling upon the tissue being examined, such that the standard procedure for conducting such examinations calls for darkening the room in which the examination is conducted. This is not only awkward but also may be impossible when the examination is conducted in rooms with large opening areas or when other procedures on other patients are being conducted in the same room served by common conventional lighting. Also, some of the incident light emitted often shines in the eyes of the person performing the examination (referred to herein as “interfering light”). This can make it difficult to see the tissue being examined.

Accordingly, a need exists for a method and apparatus of detecting abnormal epithelial tissue where interfering light is not directed toward the examiner's eyes.

Also, a need exists for a method for conducting selective light examinations that can be carried out without darkening the room in which the examination is conducted.

### SUMMARY OF THE PREFERRED EMBODIMENTS

In accordance with a preferred embodiment of the present invention there is provided a method of detecting abnormal epithelial tissue. The method includes the steps of providing a light source, providing a housing having an opening defined therein, inserting the light source into the housing, illuminating an area of epithelial tissue with incident light emitted from the light source that is directed through the opening in the housing, wherein at least a portion of the incident light is reflected from the area, thereby creating reflected light having at least one wavelength, and viewing the reflected light. The housing is preferably opaque.

In a preferred embodiment, the method further includes providing spectacles or goggles having a filter, and the step of filtering the reflected light comprises filtering the reflected light with the spectacles, thereby providing filtered light. In another embodiment, the method is performed by an examiner, and further includes manipulating the housing so that substantially none of the incident light directed through the opening is directed into the eyes of the examiner.

In accordance with another preferred embodiment of the present invention there is provided a retractor used to perform examinations for detection of abnormal epithelial tissue. The retractor includes a handle portion and a housing that is detachably connectable to the handle. The housing defines an interior and an opening that communicates the interior with an exterior of the housing when the handle and housing are connected, and the housing is opaque. In a preferred embodiment a light source is disposed in the housing and the handle portion includes a handle and an axially aligned male connection member extending therefrom. The male connection member extends into the housing through an opening in an end thereof.

In accordance with yet another preferred embodiment of the present invention there is provided a method of detecting abnormal epithelial tissue. The method includes the steps of providing a housing that includes a light source disposed therein, is opaque and has an opening defined therein, illuminating an area of epithelial tissue with incident light emitted from the light source, wherein the incident light is directed through the opening in the housing and at least a portion of the light is reflected from the area, thereby creating reflected light that has at least one wavelength, and viewing the reflected light.

### BRIEF DESCRIPTION OF THE DRAWINGS

FIG. 1 is a graph showing the preferred spectral output in accordance with an embodiment of the present invention;

FIG. 2 is a graph showing preferred lens transmission in accordance with an embodiment of the present invention;

FIG. 3 is an exploded perspective view of a retractor and chemiluminescent light source in accordance with an embodiment of the present invention;

FIG. 4 is a perspective view of the assembled retractor of FIG. 3;

FIG. 5 is a perspective view of an examination of the oral cavity using the retractor of FIG. 3; and

FIG. 6 is a cross-sectional view of the retractor of FIG. 3 taken along line 6-6 of FIG. 4.

Like numerals refer to like parts throughout the several views of the drawings.

### DESCRIPTION OF THE PREFERRED EMBODIMENTS

As shown in the drawings, for purposes of illustration, the invention is embodied in a method and apparatus for detecting abnormal epithelial tissues. The apparatus includes a retractor that emits incident light from a light source disposed therein and through an opening.

Briefly, one embodiment of the invention for screening epithelial tissue for possible abnormal tissue includes illuminating a gross anatomical area of epithelial tissue with incident light, that selectively aids in visualizing abnormal tissue sites on said gross area and viewing the illuminated gross area of tissue through a filter lens which transmits to the examiner reflected light only in certain desired wavelengths, while substantially blocking transmission of ambient or interfering light of wavelengths other than the reflected light of the desired wavelengths, thus enhancing the selective visualization of any abnormal tissue sites in the presence of normal ambient light.

For exemplary purposes only, described hereinbelow is a preferred embodiment wherein the retractor houses a chemliuminescent light source. However, this is not a limitation on the present invention. It will be understood that the method and apparatus can use any type of light source, for example, incandescent, fluorescent, and the like. Any light source that emits incident light that can be reflected back in the preferred spectral range is within the scope of the present invention. Other light sources will be readily apparent to those skilled in the relevant art.

It will be appreciated that terms such as “front,” “back,” “top,” “bottom,” and “side” used hereinbelow are merely for ease of description and refer to the orientation of the components as shown in the Figures. It should be understood that any orientation of the retractor, and the components thereof described herein is within the scope of the present invention.

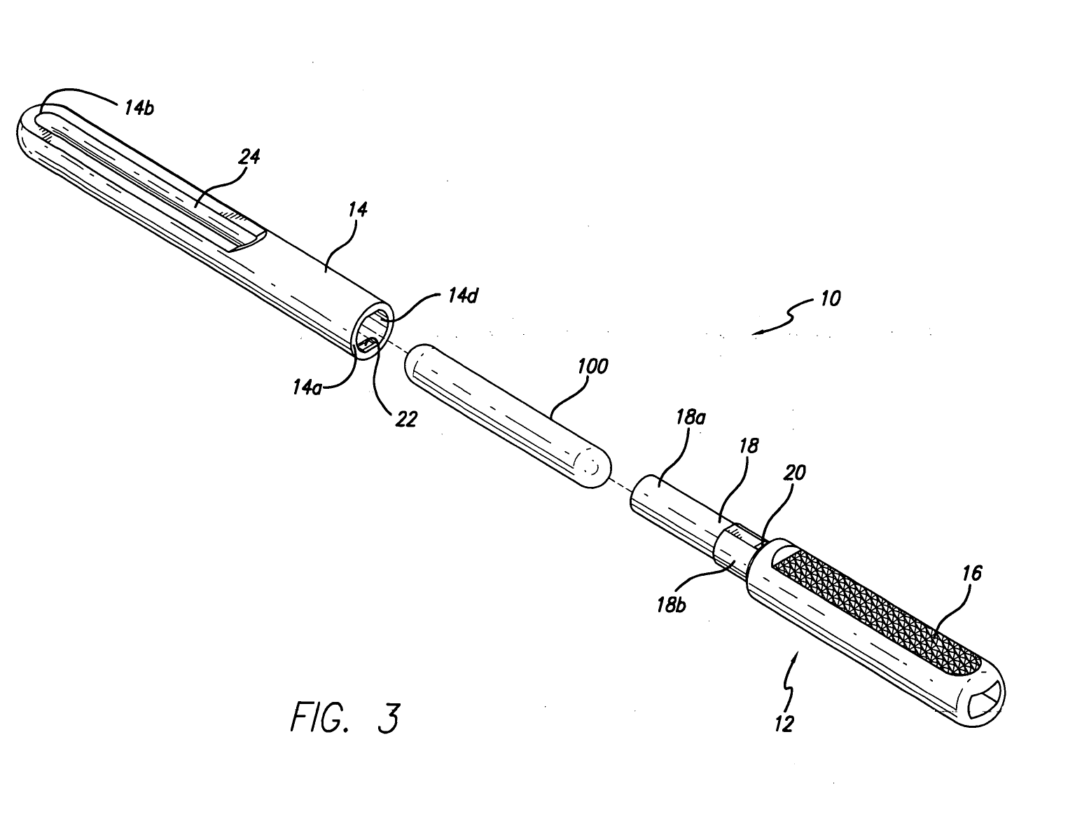

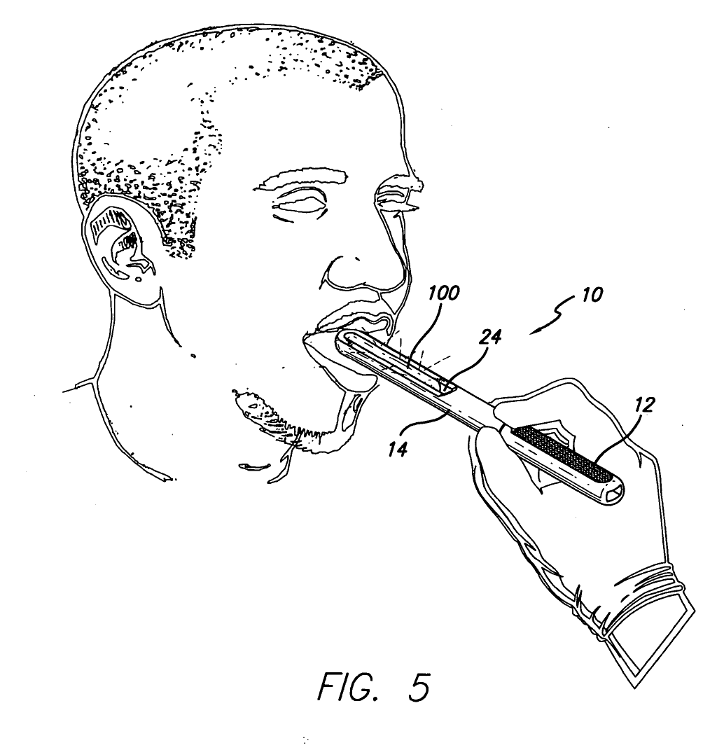

Referring to FIGS. 3-6, a retractor 10 for housing a light source 100 is shown. The retractor 10 includes a handle portion 12 and a sleeve or housing 14 that are detachably connectable. The handle portion 12 includes a handle 16 and a male connection member 18. The housing 14 includes first and second ends 14a and 14b and defines an interior 14c. The first end 14a of the housing defines an opening 14d into which the male connection member 18 can be inserted. In a preferred embodiment, the male connection member 18 includes a protrusive portion 18a and a stepped portion 18b that is sized to be received through the opening 14d in housing 14.

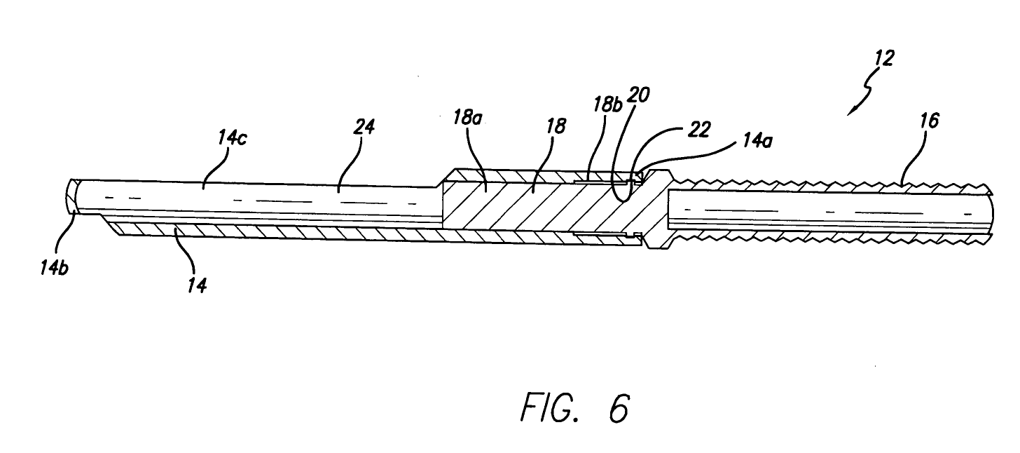

Preferably, the stepped portion 18b includes at least one tab 20 (two are shown in FIG. 6) that is received in a corresponding recess 22 defined in the inside surface of housing 14. This arrangement keeps housing 14 attached to or snap fit on the handle portion 12. To detach the two components a user simply pulls the housing 14 and handle portion 12 in opposite directions. This will disengage the tabs 20 and recesses 22 and pull the housing 14 off of the male connection member 18. In use, because the housing 14 is detachable from the handle portion 12, the housing 14 can be disposed after use.

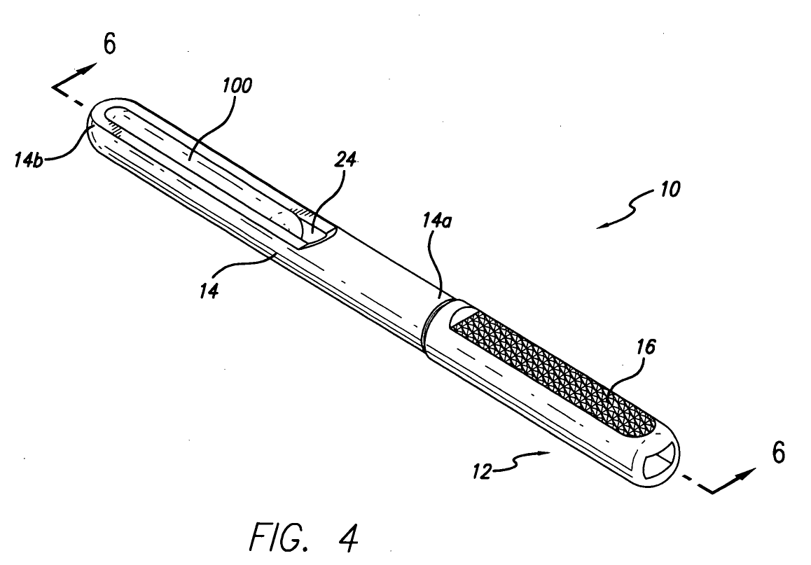

As shown in FIG. 4, the housing 14 is sized to receive a light source 100. The light source 100 is inserted into the housing interior 14c through opening 14d and then is followed by inserting the male connection member 18 through opening 14d to connect the handle portion 12 with the housing 14.

As can be seen in FIG. 3, the housing 14 has an opening 24 defined therein. In a preferred embodiment, the opening 24 is elongated as shown. It will be understood that the housing 14 is made of an opaque material. In a preferred embodiment, the housing 14 is made of a lightweight plastic, such as polystyrene. However, it will be understood that the housing 14 can be made of any material that is opaque and can be formed to house a light source. In another embodiment, the retractor 10 can be made of a transparent material.

FIG. 6 shows another opening on the side of the housing 14 opposite the opening 24 and near the second end 14b. However, this opening can be omitted.

The shape and location of the opening 24 is not a limitation on the present invention. For example, the opening can be smaller or located at the tip of the housing. Any shape and location of the opening that selectively prevents interfering light from being directed toward the examiner's eyes is within the scope of the present invention.

It will be appreciated by those skilled in the art that the spirit of the present invention is to provide a housing or sleeve that surrounds a light source and includes an opening therein for directing incident light toward the tissue to be examined. Any apparatus that directs incident light as described is within the scope of the present invention. For example, U.S. Pat. No. 6,496,718 to Lonky, incorporated herein by reference, teaches a body cavity light that uses an omni-directional diffuse light source. It is within the scope of the invention to provide a sleeve or housing that covers the diffuse light source and only allows the incident light from the diffuse light source to emanate from an opening in the sleeve or housing (thereby making it non-omni-directional). In other embodiments, the housing can be flexible or extendable. For example, in an embodiment where the housing is flexible and/or extendable a mechanism, such as a fiber through which light is transmitted (e.g., fiberoptics), for transmitting the light from the light source to the opening in the housing would preferably be included. The housing can also be disposable or sterilizable.

In another embodiment, the light source can be connected to the handle and the housing or sleeve can be removable from the handle and housing combination.

The following examples are presented to enable those skilled in the art to understand and practice the invention and to identify the presently preferred embodiments thereof. These examples are provided for illustrative purposes and not to indicate the scope of the invention which is defined only by the appended claims.

### EXAMPLE 1

A routine visual examination of the oral cavity is made, noting the presence or absence of any lesions on the attached gingiva, the buccal mucosa, the floor of the mouth, the hard and soft palate and the dorsal, lateral and ventral tongue. The presence or absence of any lesions noted by this routine examination are recorded.

### EXAMPLE 2

After completing the routine examination of Example 1, the patient is then instructed to rinse the mouth with a 1% acetic acid solution for up to one minute and then expectorate. The chemiluminescent light source described in the Lonky patent U.S. Pat. No. 5,329,938, commercially available under the registered trademark VIZILITE®, is activated by bending the flexible outer capsule, breaking the brittle inner vial. The capsule (which is light source 100) is then shaken and inserted into the housing 14 of the retractor 10. The handle portion 12 is then connected to the housing 14.

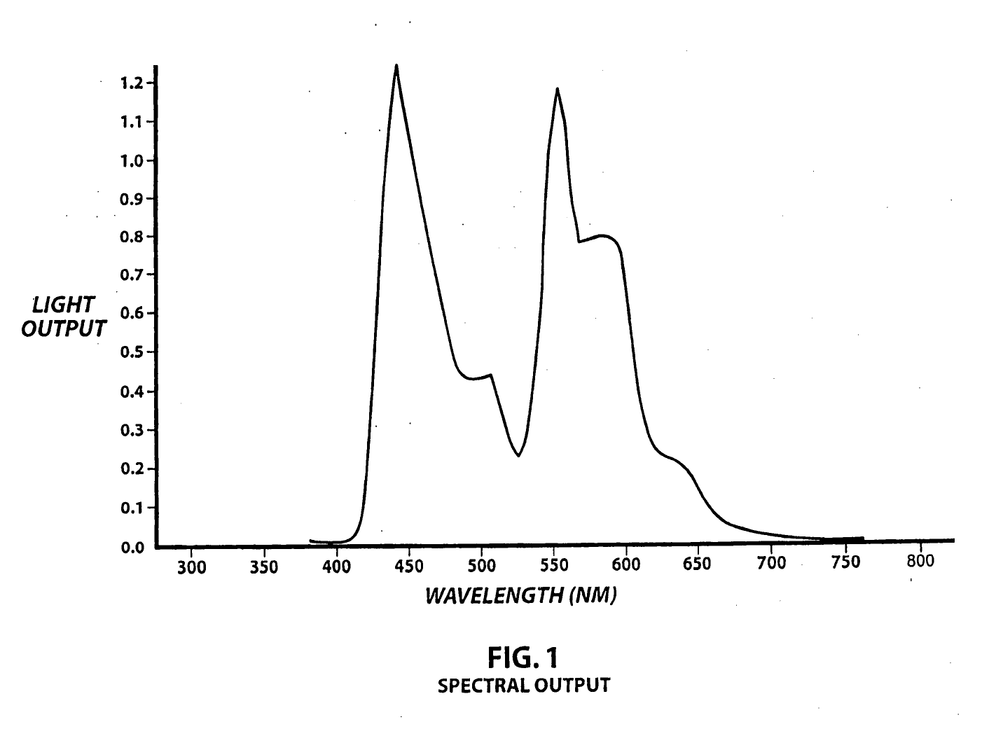

The light provided has spectral peaks at about 430 nm, 550 nm and a smaller peak in the red region at about 600 nm, as indicated in FIG. 1. These spectral peaks produce a bluish-white light.

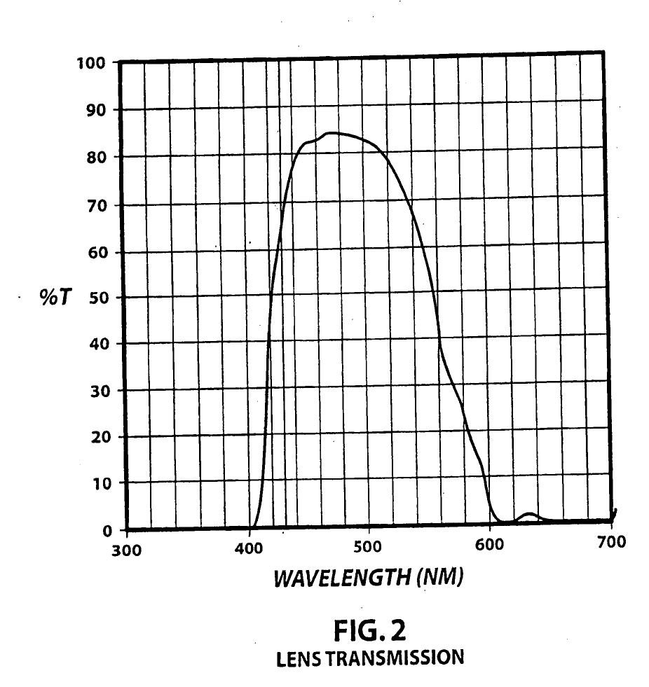

In a preferred embodiment, the examining clinician then dons a pair of spectacles provided with at least one lens which only transmits light in the wavelength band of 400-600 nm, as indicated in FIG. 2. These spectacles are shaped to minimize illumination reaching the examiner's eyes from above and from the sides.

Without reducing ambient light from normal illumination sources, the visual examination of the oral cavity is then repeated using the illumination provided by the light source, looking for lesions or other suspect tissue sites which appear white, paying special attention to any suspect tissue sites noted in the routine examination of Example 1. Any sites which appear white or bluish-white are noted and recorded.

Further assessment of the noted sites is made, for example by tissue biopsy for standard histology or by molecular analysis, to determine whether the tissue is cancerous or harbors mutations which are in the pathway for eventual development of invasive cancer.

### EXAMPLE 3

After completing the routine examination of Example 1, the patient is then instructed to rinse the mouth with a 1% acetic acid solution for up to one minute and then expectorate. The chemiluminescent light source described in the Lonky patent U.S. Pat. No. 5,329,938, commercially available under the registered trademark VIZILITE®, is activated by bending the flexible outer capsule, breaking the brittle inner vial. The capsule (which is light source 100) is then shaken and inserted into the housing 14 of the retractor 10. The handle portion 12 is then connected to the housing 14.

The light provided has spectral peaks at about 430 nm, 550 nm and a smaller peak in the red region at about 600 nm, as indicated in FIG. 1. These spectral peaks produce a bluish-white light. The light is only emitted through the opening 24, which directs the incident light toward the tissue to be examined. The remainder of the incident light (the interfering light) is blocked by the housing 14.

The retractor 10 is then inserted into the oral cavity of the patient and the visual examination of the oral cavity is then repeated using the illumination provided by the light source that is directed through the opening 24. The retractor 10 is preferably manipulated so that little or none of the emitted incident light is directed toward the eyes of the examiner before being reflected. The examination is performed looking for lesions or other suspect tissue sites which appear white, paying special attention to any suspect tissue sites noted in the routine examination of Example 1. Any sites which appear white or bluish-white are noted and recorded.

Further assessment of the noted sites is made, for example by tissue biopsy for standard histology or by molecular analysis, to determine whether the tissue is cancerous or harbors mutations which are in the pathway for eventual development of invasive cancer.

It will be understood that the present invention can be used for examination of other areas of the body. For example, the present invention can be used to perform an endoscopic examination of the esophagus, the cervix or the colon to detect early signs of cancer. In this type of examination, a sleeve or the like with an opening for emitting incident light is inserted into the patient's esophagus, cervix or colon and the examination is performed. As will be understood by those skilled in the art of endoscopy, the inside of the organ and the reflected light is viewed remotely on a monitor.

The foregoing embodiments are merely examples of the present invention. Those skilled in the art may make numerous uses of, and departures from, such embodiments and combinations of such embodiments without departing from the spirit and the scope of the present invention. Accordingly, the scope of the present invention is not to be limited to or defined by such embodiments in any way, but rather, is defined solely by the following claims.

What is claimed is:

1. A method of detecting abnormal epithelial tissue, comprising:
providing a light source,
providing a housing having an opening defined therein, wherein the housing is opaque,
inserting the light source into the housing,
illuminating an area of epithelial tissue with incident light emitted from the light source, wherein the incident light is directed through the opening in the housing, and wherein at least a portion of the incident light is reflected from the area, thereby creating reflected light having at least one wavelength, and
viewing the reflected light.

2. The method of claim 1, further comprising determining if the reflected light is white.

3. The method of claim 2, wherein if the reflected light is white, the method further comprises performing an assessment of the area, wherein the assessment is one selected from the group consisting of a tissue biopsy, a histological analysis, or a molecular analysis.

4. The method of claim 1, wherein the at least one wavelength of the reflected light is between about 400 nm and about 750 nm.

5. The method of claim 1, wherein the abnormal epithelial tissue includes tumor phenotypes.

6. The method of claim 1, wherein the illuminating step comprises directing incident light emitted from a chemiluminescent light source towards the area of epithelial tissue.

7. The method of claim 1, wherein the at least one wavelength of the incident light comprises a first wavelength of about 450 nm, a second wavelength of about 550 nm, and a third wavelength of about 600 nm.

8. The method of claim 4, further comprising filtering the reflected light to substantially remove wavelengths other than the at least one wavelength, thereby creating filtered light, and viewing the filtered light.

9. The method of claim 8, wherein the step of filtering further comprises substantially removing any ambient light not coming from the light source.

10. The method of claim 8, further comprising providing spectacles having a filter, and wherein the step of filtering the reflected light comprises filtering the reflected light with the spectacles to only allow filtered light through.

11. The method of claim 1, wherein the method is performed by an examiner, and wherein the method further comprises manipulating the housing so that substantially none of the incident light directed through the opening is directed into the eyes of the examiner.

12. A retractor used to perform examinations for detection of abnormal epithelial tissue, the retractor comprising:
a) a handle portion, and
b) a housing that is detachably connectable to the handle, wherein the housing defines an interior and an opening that communicates the interior with an exterior of the housing when the handle and housing are connected, wherein the housing is opaque.

13. The retractor of claim 12 further comprising a light source disposed in the housing.

14. The retractor of claim 13 wherein the light source is chemiluminescent.

15. The retractor of claim 12 wherein the handle portion includes a handle and an axially aligned male connection member extending therefrom, wherein the male connection member extends into the housing through an opening in an end thereof.

16. A method of detecting abnormal epithelial tissue, comprising:
providing a housing that includes a light source disposed therein, wherein the housing is opaque and has an opening defined therein,
illuminating an area of epithelial tissue with incident light emitted from the light source, wherein the incident light is directed through the opening in the housing, and wherein at least a portion of the incident light is reflected from the area, thereby creating reflected light having at least one wavelength, and
viewing the reflected light.

17. The method of claim 16, wherein the method is performed by an examiner, and wherein the method further comprises manipulating the housing so that substantially none of the incident light directed through the opening is directed into the eyes of the examiner.

18. The method of claim 16 wherein the reflected light is viewed remotely.

19. The method of claim 18 wherein the reflected light is viewed on a monitor.
